(19) United States
(12) Patent Application Publication
(10) Pub. No.: US 2005/0214377 A1
(43) Pub. Date: Sep. 29, 2005

(54) Microparticles for cell delivery

(76) Inventors: Sanjay Mistry, Downingtown, PA (US); Iksoo Chun, Princeton, NJ (US); Darin Jeffrey Messina, Downingtown, PA (US); Anna Gosiewska, Skillman, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/081,247

(22) Filed: Mar. 16, 2005

Related U.S. Application Data
(60) Provisional application No. 60/555,908, filed on Mar. 24, 2004.

(51) Int. Cl. A61K 9/14; A61K 9/16; A61K 9/50

(52) U.S. Cl. 424/489; 424/93.7; 514/12

(57) ABSTRACT

The invention relates to microparticles and compositions containing the microparticles for use in delivering viable cells to specific regions in the body for treatment of diseases in the body, and to methods of treatment of such diseases, where the microparticles include a core containing an effective amount of a biologically active agent distributed there through, and a cell attached to the surface of the core, and where the compositions contain the microparticles and a vehicle for the microparticles.

### FIELD OF THE INVENTION

The present invention relates to microparticles, devices and compositions useful in delivering cells to identified regions of the body of a mammal for the treatment of diseases, in particular, diseases caused by the absence of biological factors.

### BACKGROUND

Many diseases, especially neurological disorders, appear to be based in whole, or in part, on the absence or limited availability of critical biological factors to target cells or regions of the body. In an attempt to provide a continuous supply of drugs or other factors to the brain and other tissues at a controlled rate, miniature osmotic pumps have been used. However, limited solubility and stability of certain drugs, as well as reservoir limitations, have restricted the usefulness of this technology.

Controlled sustained release of biological factors in the body by implanting factors encapsulated within biodegradable polymeric microcapsules that do not contain cells has been attempted. However, the finite loading capacity of the polymer and the lack of any cellular feedback regulation limit the delivery of biological factors to the targeted regions.

Many investigators have attempted to reconstitute organ or tissue function by transplanting whole organs, organ tissue, or cells that provide secreted products or affect metabolic functions. Moreover, transplantation can provide dramatic benefits but is limited in its application by the relatively small number of organs suitable and available for grafting. However, the patient generally must be immunosuppressed in order to avert immunological rejection of the transplant, which results in loss of transplant function and eventual necrosis of the transplanted tissue or cells. In many cases, the transplant must remain functional for a long period of time, even for the remainder of the patient's lifetime. It is both undesirable and expensive to maintain a patient in an immunosuppressed state for a substantial period of time.

For neurological disorders, including neurodegenerative diseases like Parkinsons, Alzheimers, stroke, multiple sclerosis, amytrophic lateral sclerosis, traumatic brain injury, and spinal cord injury, the implantation of cells capable of constitutively producing and secreting neurologically active factors has also been attempted. Recently, remedial transplantation of neurotransmitter-secreting tissue has been accomplished using the patient's own tissue so as not to elicit an immune response. For example, dopamine-secreting tissue from the adrenal medulla of patients suffering from Parkinson's disease has been implanted in their striatum with some success. However, this procedure is only used in patients less than 60 years of age, as the adrenal gland of older patients may not contain sufficient dopamine-secreting cells. This restriction limits the usefulness of the procedure as a remedy since the disease most often affects older people.

A number of researchers have proposed the use of microcapsules, or tiny spheres, that encapsulate a microscopic droplet of a cell solution, for both therapeutic implantation purposes and large-scale production of biological products. There are, however, a number of shortcomings to the microencapsulation approach. For example, microcapsules can be extremely difficult to handle, including being difficult to retrieve after implantation. The types of encapsulating materials that can be used are constrained by the formulation process to polymers that can dissolve in biocompatible solvents. Furthermore, due to the limited diffusional surface area per unit volume of larger size spheres, only a limited amount of tissue can be loaded into a single microcapsule.

An alternative approach to microencapsulation has been macroencapsulation, which involves loading cells into hollow fibers and then sealing the extremities. In contrast to microcapsules, macrocapsules offer the advantage of easy retrievability, an important feature in therapeutic implants, especially neural implants. However, the construction of macrocapsules in the past has often been tedious and labor intensive. Moreover, due to unreliable closure, conventional methods of macroencapsulation have provided inconsistent results. The method is further limited to utilizing cell types that will not divide, since it has been shown that delivering cells, following placement in such a device, over expand and undergo necrosis.

Therefore, there exists a need for improved devices, compositions and methods for the treatment of neurological and other disorders in general. More particularly, there is a need for therapeutic devices and/or compositions that can augment or replace the functions of dysfunctional areas of the brain or other organs without causing excessive trauma. Thus a need exists for a device and method for providing neuroactive factors to a localized region of the nervous system of a subject, the correct dosage of which should be constitutively delivered over time to the nascent repopulating cell population.

The inventions claimed herein provide such devices, compositions and methods utilizing a microparticle that contains cells for production and delivery of critical biological factors to the target region and that also delivers biologically active agents that enhance cell survival, proliferation, and maintenance of the cells in a differentiated form. The devices can be easily and reproducibly manufactured and are in a form suitable for implantation with cells.

### SUMMARY

The invention relates to microparticles and compositions containing the microparticles for use in delivering viable cells to specific regions in the body for treatment of diseases in the body, and to methods of treatment of such diseases, where the microparticles includes a core containing an effective amount of a biologically active agent distributed there through and a cell attached to the surface of the core, and where the compositions contain the microparticles and a vehicle for delivery of the microparticles.

### BRIEF DESCRIPTION OF THE DRAWINGS

FIG. 1 is schematic cross-section of a microparticle of the present invention.

FIG. 2 is schematic cross-section of a microparticle of the present invention.

FIG. 3 is a photograph of a microparticle comprising a GDNF-loaded core with cells attached thereto.

### DETAILED DESCRIPTION OF THE INVENTION

Microparticles, as well as compositions and devices containing the microparticles, are provided where the microparticles comprise a core containing an effective amount of a biologically active agent distributed there through and cells attached to the core as described in detail herein. The presence of the core and the selection of certain components of the core provide for controlled, sustained or extended release of the biologically active agent, while avoiding premature release or degradation of the biologically active agent and/or cells prior to release.

By effective amount of a biologically active agent it is meant that the biologically active agent will be present in the core in an amount effective to enhance survival of the implanted cell and/or cells already present in the body, as well as integration and/or differentiation of the implanted cell upon release from the core. As used herein, “enhanced survival” refers to survival of cells for greater than 24 hours, up to 3 months, or sufficient time for cell integration. A sufficient amount of a biologically active agent will be released over time to allow for the implanted cells to survive and integrate into the host tissue, depending on the degree of host tissue degeneration observed.

As used herein, “microparticle” refers to a particle having a diameter of from about 1 micrometer to about 500 micrometers, preferably from about 10 to about 200 micrometers for most applications. Unless otherwise noted, the term “microparticle” is used herein to encompass both microparticulates and microspheres. Microparticulate is used herein to describe particles of irregular or non-spherical shape. Microspheres is used herein to describe substantially spherical particles. As used herein, “sustained” or “extended” release of the biologically active agent may include continuous or discontinuous release over time. The release profile may be linear or non-linear over time. The required release profile may be selected depending on the particular disorder being treated.

When determining and selecting an appropriate release profile, one may consider the structure of the microparticle, as well as the composition of the core with respect to its dissolution or degradability once placed in the body. For example, the core may comprise blends and/or layers of materials as carriers, whether the same or different, and/or coatings used in preparing the microparticles in order to fashion the desired release profile. In addition, the inclusion of the biologically active agent itself, whether due to the concentration of the biologically active agent and/or the means of its incorporation in the core, may contribute to the release profile. One skilled in the art of providing for treatment requiring such controlled release will be able to ascertain the appropriate release profile once having the benefit of this disclosure.

The core may comprise the biologically active agent in combination with other ingredients or materials, such as biocompatible, pharmaceutically suitable carriers and/or processing excipients, to aid in formation and maintenance of the core and/or administration or release of the microparticles and/or biologically active agent to the body. In certain embodiments of the present invention, the core may consist essentially of the biologically active agent.

In certain embodiments of the present invention, the core will contain a biocompatible, biodegradable, continuous polymeric phase having the effective amount of the biologically active agent dispersed there through. By biodegradable, it is meant that the polymers are degraded or broken down under physiological conditions in the body such that the degradation products may be excreted or absorbed by the body. The polymeric phase serves as a carrier or reservoir for the biologically active agent. The polymer used as the carrier will be dissolved or degraded by physiologic fluids once administered in the body such that the biologically active agent may be released in a controlled, e.g. sustained or extended, manner upon exposure of the core to physiologic fluids.

The biologically active agent may be insoluble in the polymer used to prepare the carrier phase of the core. In such embodiments, the polymer may be melted and, if stable at the polymer melt temperature, the particles of the biologic agent dispersed through the molten polymer by means disclosed herein, so as to provide a substantially homogeneous dispersion of the solid biologically active agent particles through the polymer melt. Upon cooling of the molten polymer containing the dispersed biologically active agent, a solid, continuous polymeric phase comprising the biologically active agent dispersed there through is provided.

In other embodiments, the biologically active agent may be soluble in the polymer used to make the carrier phase. In such embodiments, providing that dissolving the biologically active agent does not detrimentally affect its therapeutic efficacy, the polymer may be melted, the agent dissolved in the molten polymer and the two mixed so as to provide a homogenous distribution of the agent through the polymer melt. Upon cooling, the core is provided having the agent homogenously mixed through the polymer carrier phase.

The microparticles of the present invention further comprise a cell(s) attached to the surface of the core of the microparticle. Upon implantation in the body, the biologically active agent is administered to the cell attached to the microparticle as well as released into the body in a controlled manner as described herein. The biologically active agent is selected to improve the viability of cells delivered to the targeted site, as well as cells already present at the targeted site, such as sites of neurological damage, thus providing host cell preservation/protection as well as protection of the delivered cells. Improved viability will ultimately increase the probability of cell survival, integration and differentiation once placed in the body.

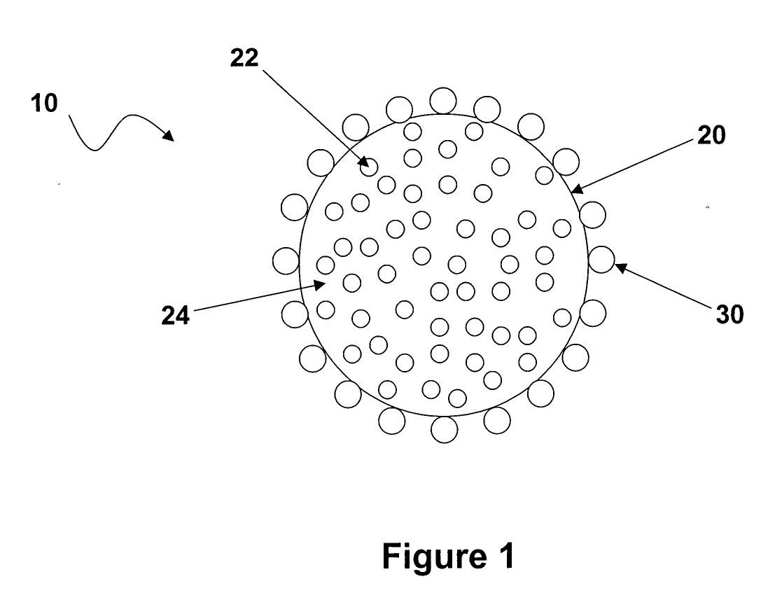

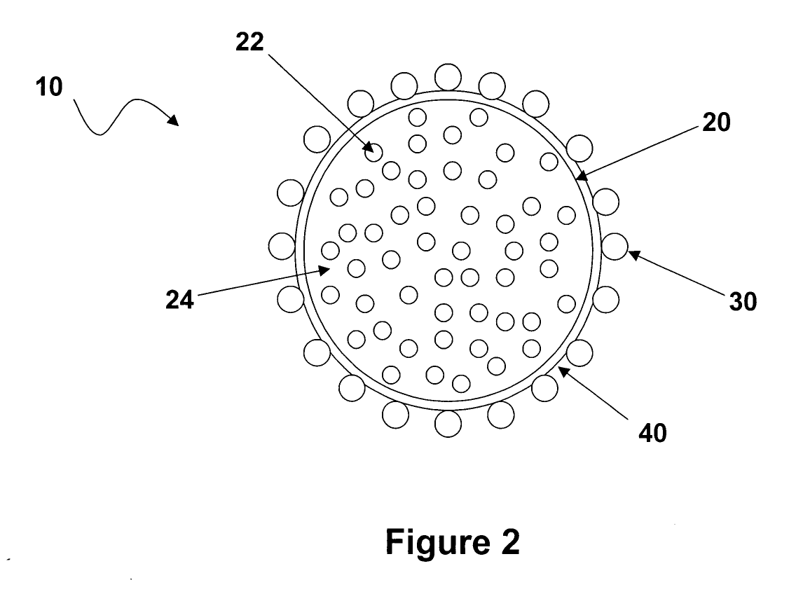

Schematic cross-sections of microparticles of the present invention are shown on FIGS. 1 and 2. FIG. 1 shows microparticle 10 containing core 20 comprising biologically active agent particles 22 substantially homogenously dispersed throughout pharmaceutically suitable polymeric carrier phase 24, and cells 30 attached to core 20. FIG. 2 shows another embodiment where core 12 is coated with cell-adherent agent 40 to assist adherence of cells 30 to core 20. While core 20 and biologically active agent 22 are shown as spherical particles, one skilled in the art could envision core 20 and/or biologically active agent 22 as being non-spherical in shape.

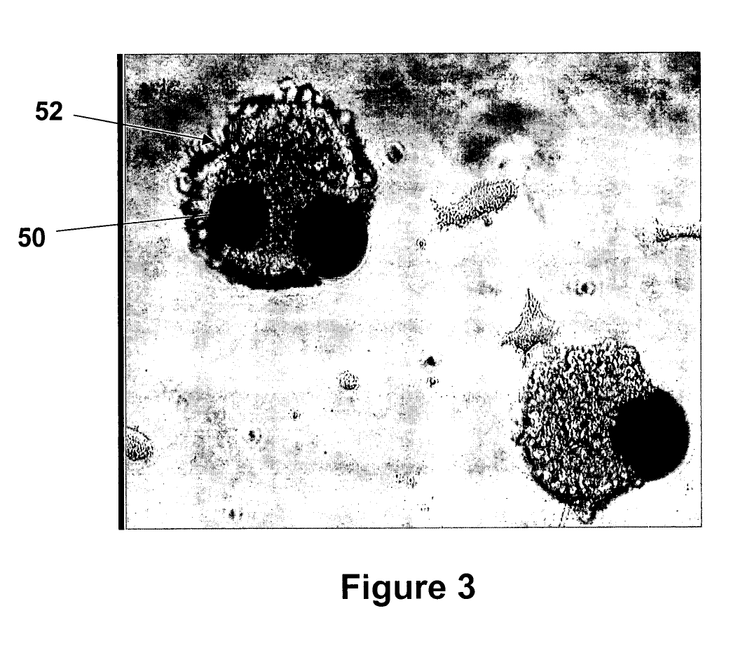

FIG. 3 is a photograph showing postpartum cells 52 attached to core 50. Core 50 comprises biologically active agent GDNF dispersed in a polymeric carrier phase comprising a 90:10 copolymer of poly-(D,L-lactic-co-glycolic) acid, as described in the examples below.

The core of the microparticle may contain from about 0.01 percent by weight up to approximately 30 percent by weight of the biologically active agent, with a preferred range of about 5 to about 10 percent. The microparticles provide sustained, controlled release of the biologically active agent from the core over a period of from at least 24 hours up to two years. In general, the microparticle may be fabricated such that the biologically active agent is released over a period of between about one month and one year.

In certain embodiments of the present invention, the diameter of the microparticle is small enough to pass through an injection needle typically used to administer therapeutic agents to the body, for example less than about 200 microns. For certain applications such as the treatment of osteogenic disorders, the diameter of the microparticles may range up to about 500 microns in diameter. For neurological disorders, microparticles may have a diameter of less than about 100 microns, or less than about 80 microns, and are suitable for administration by injection intracranialy via a micro catheter. For example, injection through a 27-gauge needle would require microparticles to be less than 100 microns in diameter.

As noted above, either natural or synthetic polymers are preferred as the pharmaceutically suitable carrier phase for the biologically active agent, although synthetic polymers are preferred due to their reproducibility both with respect to manufacture and controlled release kinetics. Synthetic polymers that can be used to form the core of the microparticle include biodegradable polymers such as poly(lactide) (PLA), poly(glycolide) (PGA), poly(lactide-co-glycolide) (PLGA), poly(caprolactone) (PCL), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, synthetic polyamino acids and prolamines; polysaccharides such as alginate, heparin; and other naturally occurring biodegradable polymers of sugar units. Alternately, combinations of the aforementioned polymers can be used.

PLA, PGA and PLA/PGA copolymers are particularly useful for forming cores used in microparticles of the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing poly(lactide)s are well documented in the patent literature.

Both the release of the biologically active agent and the biodegradability of the polymer are related to the molecular weights of the polymers, e.g. PLA, PGA or PLA/PGA. Higher molecular weights, e.g. weight average molecular weights of about 90,000 or higher, result in polymer matrices which retain their structural integrity for longer periods of time and thus provide for more extended release of biologically active agent; while lower molecular weights, e.g. weight average molecular weights of about 30,000 or less, result in faster release of the biologically active agent.

The release of the biologically active agent from the microparticle core comprising a polymeric carrier can occur by two different mechanisms. The agent can be released in the appropriate dosage form by diffusion through aqueous-filled channels generated by the dissolution of the agent once the microparticle is placed in the body, or by voids in the core created by the removal of the polymer solvent during the original formation of the core via microencapsulation of the agent. The second mechanism provides release due to the degradation of the polymer.

The degradation of certain polymers occurs by spontaneous hydrolysis of the ester linkages on the backbone. Thus, the rate of release of the biologically active agent can be controlled by properly selecting polymer properties influencing water uptake. These include, without limitation, the monomer ratio (lactide to glycolide), the use of L-lactide as opposed to D/L lactide, and the polymer molecular weight. These variables determine the hydrophilicity and crystallinity that ultimately govern the rate of water penetration. Hydrophilic excipients such as salts, carbohydrates and surfactants can also be incorporated into the core to increase water penetration into the microparticles and thus accelerate the biodegradation of the polymer.

By properly selecting the properties of the polymer and the properties of the biologically active agent, one can control the contribution of each of these release mechanisms and thus alter the release rate of the biologically active agent as necessary. Slowly-degrading polymers, such as poly(L-lactide) or high molecular weight poly(lactide-co-glycolide) copolymers having low glycolide, content will cause the release of the biologically active agent to be controlled more by diffusion. Increasing the glycolide content and decreasing the molecular weight enhances both water uptake and the hydrolysis of the polymer and introduces a polymer degradation factor to the release kinetics.

The release rate can also be modified by varying the concentration of the biologically active agent within the core of the microparticle. Increasing the concentration of the biologically active agent will increase the network of interconnecting channels formed upon the dissolution of the agent once the microparticle is placed in the body and enhance the release of the agent from the core.

In addition to factors attributable to selection and concentration of the polymers and biologically active agents, additives can be used to modify the release rate and the therapeutic stability of the biologically active agent. Polymer hydrolysis is accelerated at acidic or basic pH, so the inclusion of acidic or basic excipients can be used to modulate the rate of degradation of the polymer. The excipients can be added to the core as particulates, can be mixed either as a solid or liquid with the biologically active agent prior to incorporation of the agent into the core, or can be dissolved within the molten polymer prior to formation of the core.

One class of excipients includes materials that enhance degradation of the polymer phase upon placement of the microparticles in the body. The amount of such materials added may range between about 0.1 and about 30 weight percent, based on the weight of the polymer phase. Types of such materials include, without limitation, inorganic acids such as ammonium sulfate and ammonium chloride, organic acids such as citric acid, benzoic acids, heparin, and ascorbic acid; inorganic bases such as sodium carbonate, potassium carbonate, calcium carbonate, zinc carbonate, and zinc hydroxide; organic bases such as protamine sulfate, spermine, choline, ethanolamine, diethanolamine, triethanolamine; and surfactants such as polyoxyalkylene ether sold under the tradename PLURONIC (BASF, Mount Olive, N.J.) and substituted sorbitan derivative surfactants sold under the tradename TWEEN (ICI Americas Inc., Bridgewater, N.J.).

Excipients that modify the solubility of the biologically active agents include salts. Complexing agents, e.g. albumin or protamine, can be used to control the release rate of the protein-based biologically active agents. Stabilizers for protein-based biologically active agents include, but are not limited to, carbohydrates such as sucrose, lactose, mannitol, dextran, and heparin; proteins such as albumin and protamine; amino acids such as arginine, glycine, and threonine; surfactants such as TWEEN and PLURONIC; salts such as calcium chloride and sodium phosphate; and lipids such as fatty acids, phospholipids; and bile salts.

The weight ratios of excipient to protein-based biologically active agents are generally 1:10 to 4:1 in the case of carbohydrate to protein, amino acids to protein, protein stabilizer to protein, and salts to protein; 1:1000 to 1:20 in the case of surfactant to protein; and 1:20 to 4:1 in the case of lipids to protein.

A variety of biologically active agents can be incorporated into the core of the microparticles of the present invention. In one embodiment, microparticles are used to treat osteogenic, cardiovascular, vascular, diabetic and wound healing disorders. In these instances, useful biologically active agents 22 include, but are not limited too, basic fibroblast growth factor (bFGF), alpha fibroblast growth factor (FGF), heparin binding growth factor (hbgf), transforming growth factor alpha or beta (TGFβ), epidermal growth factor (EGF), insulin derived growth factor (IGF), vascular endothelium growth factor (VEGF), platelet derived growth factor (PDGF), glial growth factor, atrial natriuretic peptide (ANP) and brain natriuretic peptide (BNP).

For neurological disorders, biologically active agents are neuroprotectives and include, but are not limited to, glial-derived neurotrophic factor (GDNF), glial growth factor, cAMP, nerve growth factor (NGF), brain-derived neurotrophic factor (BDNF), platelet derived growth factor (PDGF), growth differentiation factor 5 (GDF5), morphogenetic protein 52, bone morphogenetic protein 7 (BMP7), bone morphogenetic protein 14 (BMP14), EPO or EPO-mimetibody, cyclosporine, and a sulfamate-substituted monosaccharide.

A variety of cells 30 can be implanted into the body using the microparticles of the present invention. The cells may be autologous or may be derived allogeneically. Where autologous cells are used considerations of immunological response by the body are minimized or eliminated, while using allogeneic cells may require further consideration of such immunological response by the body. Such a treatment strategy may require adjunct immunotherapy with cyclosporine, FK-506 or other such immunotherapeutic agents. In certain embodiments, cells utilized according to the present invention are derived in vitro under aseptic conditions utilizing conventional methods for dissociating cells from organs or tissues in vivo. Chondrocytes, angioblasts, myoblasts, epithelial cells, smooth muscle cells, keratinocytes, beta cells, sertoli cells, macrophages, microglia, and endothelial cells may be utilized for the treatment of specific diseases. Alternatively, cells may be obtained through a point of care approach.

For neurological disorders, cells that may be utilized according to the present invention include, but are not limited to, undifferentiated or pre-differentiated stem or progenitor cells, neural stem or neural progenitor cells, neural cells, dendritic cells, genetically transformed cells. Stem cells include, without limitation, hematopoietic, mesenchymal, postpartum, pancreatic, hepatic, retinal epithelial, olfactory bulb, endothelial, muscle, adipose-derived, ileac crest, bone marrow, oval and dermal stem cells.

A variety of techniques may be used to incorporate the biologically active agent into the core 20 of microparticles of the present invention. Examples are set forth below.

In spray drying, a carrier phase comprising a biocompatible, biodegradable polymer, and the biologically active agent are mixed together in a solvent for the polymer. The solvent is then evaporated by spraying the solution, leaving polymeric droplets containing the biological active agent. Spray drying techniques are reviewed in detail by K. Masters in “Spray Drying Handbook” (John Wiley & Sons, New York 1984); and Patrick B. Deasy in “Microencapsulation and Related Drug Processes” (Marcel Dekker, Inc., New York 1984). As spray drying may result in some loss of activity of the agent due to the heat generated in the process, or in loss of material due to sticking of the polymer to the chamber, it is not preferred for use when using labile materials which are available only in small quantities.

In other embodiments, solvent evaporation techniques can be used to form the core of the microparticle. These techniques involve dissolving a carrier comprising a biocompatible, biodegradable polymer in an organic solvent that contains either dissolved or dispersed solid biologically active agent. The polymer/agent solution is then added to an agitated continuous phase that usually is aqueous to form an oil-in-water emulsion. Emulsifiers are included in the aqueous phase to stabilize the oil-in-water emulsion. The organic solvent is then evaporated over a period of several hours or more, thereby depositing the polymer around the agent. Solvent can be removed from the core of the microparticle in a single step under reduced pressure, or by the application of heat. Freeze-drying may also be used to remove the solvent from the core of the microparticle.

In still other embodiments, phase separation techniques can also be used to form the core of the microparticle. These techniques involve the formation of a water-in-oil emulsion or oil-in-water emulsion. A carrier comprising a biocompatible, biodegradable polymer is precipitated from the continuous phase of the emulsion onto the biologically active agent by a change in temperature, pH, ionic strength, or the addition of precipitants. For example, U.S. Pat. No. 4,675,800 describes the formation of poly(lactic-co-glycolic) acid microspheres containing active proteins. The protein is first dissolved in the aqueous phase of a water-in-oil emulsion or dispersed as a solid in the polymer phase. Polymer is then precipitated around the aqueous droplets or drug particles by addition of a non-solvent for the polymer, such as silicone oil. The final product, as with most phase separation techniques, is in the form of a microcapsule. Microcapsules contain a core material surrounded by a polymer membrane capsule.

A method for making the core of the microparticle containing biologically active agents having the desired characteristics is described in U.S. Pat. No. 5,019,400 to Gombotz, et al, the disclosure of which is incorporated herein as if set forth in its entirety. The method disclosed therein involves rapid freezing, followed by solvent extraction.

There are two principal embodiments of the system for making cores. The first utilizes a combination liquefied gas-frozen non-solvent system. The second utilizes a frozen non-solvent system.

In the first instance, a carrier comprising a biocompatible, biodegradable polymer and a biologically active agent to be encapsulated in solution are atomized using an ultrasonic device to form a liquefied gas, e.g. liquid nitrogen. The atomized cores freeze when they contact the liquefied gas, forming frozen spheres. These sink to the surface of the frozen non-solvent, e.g. ethanol. The liquid gas is evaporated and the spheres begin to sink into the non-solvent as the non-solvent thaws. The solvent in the spheres is extracted into the non-solvent to form microspherical cores containing the encapsulated agent. Other non-solvents such as hexane may be added to the non-solvent to increase the rate of solvent extraction from certain polymers, where appropriate, e.g. when spheres are formed of polylactide-co-glycolide polymers.

Alternatively, a cold non-solvent for the polymer can be substituted for the combination of liquefied gas-frozen non-solvent system described above, provided the temperature of the non-solvent is below the freezing temperature of the polymer/agent solution. It is important to select a solvent for the polymer having a higher melting point than the non-solvent for the polymer so that the non-solvent melts first, allowing the frozen microspherical cores to sink into the liquid where they later thaw. If a cold liquid non-solvent system for making the polymeric microspherical cores is used, the cores will sink immediately into the non-solvent. As the solvent in the cores thaws, it is extracted into the non-solvent. The solvent for the polymer and the non-solvent for the polymer must be miscible to allow extraction of the solvent from the microspheres.

Cells may be attached to the core in either a non-releasable or releasable state. In a non-releasable state, the cell will remain attached to the core until such time as the core is substantially biodegraded by physiological fluids of the body, at which time the cell may be released from the core. In a releasable state, the cell may be released from the core prior to the biodegradation of the core by physiological fluids of the body, thus providing for mobility of the cell and further interaction with body tissue and cells in the region being treated. Cell attachment to the core is related to the hydrophilicity or hydrophobicity of the polymeric carrier or biologically active agent comprising the core.

On the one hand, attachment of the cell to the surface of the core may be accomplished or enhanced by the deposition onto the surface of the core of the microparticle of an extracellular matrix (ECM) that is produced by the cell. In this way, no pre-treatment of the microparticle is required to provide for attachment of the cell to the core. Thus, ECM made by a cell can include but is not limited to proteoglycans, cell-adhesion molecules such as neural-cell adhesion molecule, integrins, cytokeratins, keratins, glycosaminoglycans and anchor proteins. The synthesis of these type of cellular ECM factors can be induced by incubation with various growth factors including but not limited to FGF or EGF.

Attachment of the cell to the core also may be enhanced by the deposition of a cell-adherent material onto the surface of the core. By cell-adherent material, it is meant that the material provides for enhanced attachment of the cell to the surface of the core. Cell-adherent materials may include biocompatible synthetic or naturally-occurring materials, including the ECM described herein above, as well as collagen, laminin, fibronectin, gelatin and allylamines. In lieu of full sequence proteins, oligopeptide sequences derived from the aforementioned proteins may also be used to stimulate attachment/differentiation of the cells to the outer surface of the microparticle. Such sequences are well established and have been shown to function in a similar manner to the full sequence native proteins. These sequences could include for example the YIGSR sequence from the B1 chain of laminin, IKVAV from the A chain, or RGD sequence from fibronectin.

The cell-adherent material may be applied to the microparticle prior to attachment of the cell to the core. In one instance, the microparticles are immersed in a solution of the cell-adherent material under aseptic conditions for up to 24 hours to provide a coating of the material on the surface of the core. Alternatively, an ECM such as cationic polymer, including, but not limited to, allylamine may be sprayed onto microparticles, or an ECM such as laminin may be hydrolyzed into the particles on the outer core. Additionally, nanofiber based on self-assembling peptides may be coated on the core.

Microparticles of the present invention may be implanted in vivo via direct injection of a composition comprising the microparticles and a biocompatible, pharmaceutically acceptable vehicle for the microparticles into the affected area or via intracatheterization. Thus, both invasive and non-invasive methods may be used for delivery of microparticles according to the present invention. The concentration of the biologically active agent used in this case will depend upon the effective dose required for its intended therapeutic use upon implantation, as well as the mode of administration to be used. The dose used should be effective to achieve local, target tissue concentrations of active ingredient that are efficacious in providing the therapeutic affect sought. Effective doses may be extrapolated from dose-response curves derived from in vitro or animal model test systems, prior to the addition of cells.

For administration, a plurality of microparticles generally will be mixed prior to administration with a non-toxic, pharmaceutically acceptable vehicle substance, e.g. aqueous solutions such as normal saline or phosphate-buffered saline, and may be administered using any medically appropriate procedure, e.g. intravenous or intra-arterial administration, intrathecal injection, or intra-cerebroventricular. In addition, intracavity or direct administration via a syringe and hypodermic needle to the affected tissue may be utilized in a manner such that microparticles are not capable of being digested or eliminated by elements of the immune or lymphatic system. An example of this would be the direct injection of a mixture of microparticles and phosphate-buffered saline directly into the caudate-putamen or substantia nigra pars compacta region of the brain to ameliorate the symptoms of Parkinson's disease.

For specific delivery within the central nervous system (CNS), intrathecal delivery can be used with, for example, an Ommaya reservoir in accordance with known techniques. See, e.g., F. Balis & D. Poplack, Am. J. Pediatric. Hematol. Oncol. 11(1):74-86. (1989). Furthermore, the described delivery procedures could also be used to deliver the microparticles of the present invention to other regions of the body for treating other medical disorders.

Alternatively, microparticles could be administered in a semi-solid state. In this instance, microparticles would be mixed with a pharmaceutically acceptable vehicle as before. The resulting solution is then centrifuged, for example for 5 minutes at 150 xg, to create a semi-solid pellet of microparticles. This semi-solid pellet of microparticles would be added to an insertion cannula device containing an inner stylet. The device would be inserted into the location of interest in vivo, to the appropriate depth. The insertion cannula would then slide back to a stop position, depositing the semi-solid pellet of microparticles in the tissue. Then the cannula device would be removed from the tissue, leaving behind the semi-solid pellet of microparticles.

It should be clear that various modifications of the present invention can be made without departing from the spirit or scope of the invention. For example, the present invention should not be read to require, or be limited to, a particular material, agent, or cell line described by way of example or illustration set forth below.

### EXAMPLE 1

### Microspherical Core Preparation and Characterization

Microspherical cores containing PDGF-BB ((Chiron Corporation, Emeryville, Calif.)) were prepared by a double-emulsion technique. In brief, 50 mg of a 90:10 copolymer of poly-(D,L-lactic-co-glycolic) acid (sold under the tradename MEDISORB, Alkermes, Inc., Wilmington, Ohio), with an intrinsic viscosity of 0.4 dl/gm, was dissolved in 1.5 ml of methylene chloride and 0.5 ml of acetone. 60 micrograms of PDGF-BB was dissolved in 0.15 ml of 16 mM citrate buffer (pH 6.0) containing 0.1 percent (wt./vol.) HAS, or human serum albumin (Sigrna, St, Louis, Mo.). PDGF-BB had been reconstituted with excipients to stabilize the protein prior to incorporating it into the microspheres. In other experiments HSA was not included. The polymer/PDGF-BB aqueous solution was sonicated continuously using a Branson Sonifier 450 (Branson Ultrasonics, Danbury, Conn.) by pulsing for 10 seconds to yield a single emulsion. The single emulsion was added into 30 ml of an aqueous solution containing 5 percent (wt./vol.) polyvinyl alcohol (MW 31,000-50,000, 87-89 percent hydrolyzed; Aldrich Chemical Company, Milwaukee, Wis.) and 5 percent (wt./wt.) NaCl. The resulting solution was magnetically stirred for 1 minute, which generated a double emulsion. This double emulsion (water/oil/water) was added to deionized water (400 ml) containing 10 percent (wt./wt.) NaCl, and magnetically stirred for 25 minutes. The microspherical cores were then filtered through a 40-micron nylon cell strainer (Becton Dickinson, NJ) and washed with deionized water (400 ml). The cores were then frozen for 2 hours at −80° C. Microspheres were then freeze-dried by a lyophilization process using a Virtis Freezemobile (Virtis Company, Gardinier, NYC). Microspheres were lyophilized retaining an ambient temperature of −40° C. The freeze-dried microspherical cores were stored in sterile microvials at 4° C. until further use.

Control cores containing no biologically active agent were prepared using the same procedure used to form the agent containing cores, except that no agent was added during the formation of the single emulsion. Core sizes for drug loaded and non-loaded microspheres were determined to be between 20 and 100 microns in size.

The release of PDGF from the microspheres was determined by ELISA. 1 mg of microspheres was placed in 0.5 ml growth medium in a 24 well cell culture plate for 24 and 168 hours. At each time point, 5 microlitres of growth medium was collected and stored at −20° C. until ELISA testing. The release of PDGF from the microspheres was determined to be 31 percent and 72 percent at 24 and 168 hours, respectively. 1 mg of microspheres was estimated to contain 3.1 micrograms of PDGF. Thus, at 24 and 168 hours it was demonstrated that 0.96 micrograms and 2.23 micrograms of PDGF were released, respectively. When adult postpartum or neural progenitor cells were attached to these particles, a dramatic increase in cell proliferation was observed, suggesting that the PDGF was stable and active. Compared to controls (microspheres with no drug) a 4-6-fold increase in cell proliferation was observed in cells attached to PDGF loaded microspheres.

In addition, PDGF release was measured by HPLC from microparticles created through both emulsion and dispersion methods. Briefly, 10 grams of a 85:15 copolymer of poly-(D,L-lactic-co-glycolic) acid (sold under the trade name MEDISORB, Alkermes, Inc., Wilmington, Ohio) was dissolved in 240 grams of methylene chloride. Two emulsions of PDGF with excepients were prepared with two different loadings, 9.2 milligrams and 18.74 milligrams, respectively, in 5 grams of a 16 millimolar citrate buffer solution (pH 6). Each emulsion was mixed with the PLGA polymer solution and sonicated. The solution was poured into a 3-inch diameter rotated disk with the disk temperature at ambient temperature, at 3000 rpm. Solution feed rates were 191 grams/minute and 174 grams/minute. Temperatures of the top cone and the bottom cone were 61° C. and 46° C., respectively.

For microparticles created through dispersion methods, 10 grams of a 85:15 copolymer of poly-(D,L-lactic-co-glycolic) acid (sold under the trade name MEDISORB, Alkermes, Inc., Wilmington, Ohio) was dissolved in 240 grams of methylene chloride. PDGF with excepients were dispersed in the PLGA polymer solution and sonicated. Two different loadings, 9.2 milligrams and 19.21 milligrams, respectively, were used. The solution was poured into a 3-inch diameter rotated disk, with the disk temperature at ambient temperature, at 3500 rpm and 3000 rpm. Solution feed rate was 188 grams/minute and 167 grams/minute. Temperatures of the top cone and the bottom cone were 61° C. and 46° C., respectively.

HPLC was used to measure recovery of PDGF from the microspheres. 100 milligrams of microspheres were placed in 0.5 milliliters of 70% acetonitrile and 0.1% TFA. The solution was mixed at room temperature overnight. Samples were diluted at 1:10 with mobile phase A before injection to HPLC. Recovery of PDGF was greater than 66% for both emulsion formulations, while dispersion methods yielded excellent recovery rates above 95%.

### EXAMPLE 2

### Attachment of Adult Postpartum Cells to GDNF-Loaded Microspheres

Postpartum stem cells were isolated from explants of postpartum tissue. The tissues were obtained from a pregnancy at the time of parturition or a normal surgical delivery. The following cell isolation protocols were performed under aseptic conditions in a laminar flow hood. The postpartum tissues were washed in phosphate buffered saline (PBS) in the presence of antimycotic and antibiotic (AA) (1 milliliter per 100 milliliter (10,000 Units per milliliter)) (PBS-AA). The washing step consisted of rinsing the tissue with PBS-AA using gentle agitation. This process was performed several times to remove blood and debris. The washed tissues were then mechanically dissociated in 150 cm tissue culture plates in the presence of 50 milliliter of DMEM-Low glucose (DMEM:Lg) or DMEM-high glucose (DMEM:Hg) medium. Once the tissues were chopped into small pieces, they were transferred to 50-milliliter conical tubes with approximately 5 gm of tissue per tube. The tissue was then digested in 40 milliliters DMEM:Lg or DMEM:Hg containing AA with 10 milliliters of collagenase:dispase (C:D) dissolved in DMEM or collagenase:dispase:hyaluronidase (C:D:H) dissolved in DMEM. C:D was 750 milligram of collagenase type II (>125 Units per milligram (0.5-3 FALGA Units per milligram)) with 500 milligram dispase (0.4 Units per milligram) diluted in 50 milliliters of DMEM. Thus, C:D:H was 750 milligram of collagenase type II (>125 Units per milligram (0.5-3 FALGA Units per milligram)) with 500 milligram dispase (0.4 Units per milligram) with 200 milligram (300 Units per mg) diluted in 50 milliliter of DMEM. Alternatively collagenase type IV (750 milligram at >125 Units per milligram (0.5-3 FALGA Units per milligram)) was also utilized in this protocol. The conical tubes containing the tissue, medium and digestion enzymes were incubated in an orbital shaker (medium shaking) at 37° C. for less than 24 hours. After digestion the tissues were filtered with 40-micrometer nylon cell strainers. The filtered cell suspensions were then centrifuged at 1000×g for 10 minutes. The supernatant was aspirated and the cell pellet resuspended in 50 milliliters of fresh medium. This process was completed twice to remove residual enzyme activity from the cell populations. Supernatant was then removed and the cell pellets were resuspended in 2 milliliters of expansion medium (DMEM:Lg or DMEM:Hg; 15 percent FBS (Hyclone Defined bovine serum Lot#AND18475); 2-mercaptoethanol (1 microliter per 100 milliliters); antibiotic per antimycotic (1 milliliter per 100 milliliters (10,000 Units per milliliter)). Cell viability per numbers of cells isolated was determined by a manual count of trypan blue exclusion.

The cells were culture-expanded for 3 weeks, with cells being passaged weekly. Freeze-dried microspherical cores prepared in Example 1 were washed several times in tissue culture medium to remove debris. 20-50 cores were mixed with 500,000 postpartum cells in a sterile eppendorf (Ambion, Austin, Tex.) in 1 ml of medium for 10-30 minutes. In this series of experiments some cores were coated with laminin (ECM) prior to mixing them with postpartum cells. This core/cell suspension was seeded into 2 wells (500 microlitres/well) of a 24 well ultra low cluster tissue culture plate (Corning, N.Y.). 100 microlitres of fresh medium was supplemented into each well for an additional 5 days. FIG. 3 shows postpartum cells 52 attached to a GDNF-loaded core 50.

In other experiments, in order to assess cell viability/phenotype, microspherical cores were removed from the culture plate and centrifuged for 3 minutes at 800 rpm. The supernatant was removed and the pellet resuspended in fresh 4 percent para-formaldehyde for a period of 20 minutes at room temperature. After this time, cores with cells attached were centrifuged once again and resuspended in fresh 1× phosphate-buffered saline (PBS, GibcoBRL). Fixed samples were then placed in a small dish and flash-frozen within OCT embedding compound (Polysciences, Warrington, Pa.) on a dry-ice bath in preparation for cryostat sectioning. A standard cryostat was used to cut sections of 10-micron thickness. Consecutive sections were placed on glass microscope slides (VWR) and further processed using 4′,6-diamidino-2-phenylindole,dihydrochloride (DAPI; Molecular Probes) as a counter-stain to label cell nuclei. DAPI was applied for 10 minutes at a concentration of 10 mM and washed with PBS after application.

Cells attached within 24 hours and were demonstrated to remain viable several days after attaching to the microspherical cores. To determine cell viability after 24 hours of attachment to the cores, cells were released from the cores by trypsinizing with trypsin-EDTA (Invitrogen, CA). The trypsinized cells were separated from the cores by passing through a 40-micron nylon cell strainer (Becton Dickinson, NJ). Cell viability was determined using the trypan-blue exclusion test. After 24 hours, 100 percent of the attached cells were demonstrated to be viable. Furthermore, cells were trypsinized from these GDNF microspheres 40 days after attachment and were shown to be viable. When these cells were replated, they were demonstrated to expand to confluence confirming their viability and proliferative potential following attachment to the drug loaded microspheres.

### EXAMPLE 3

### Implantation of Microspheres into the Striatum

50:50 and 85:15 PLGA microspheres were prepared by dissolving the polymer in methylene chloride. A phase separation agent was added during the stirring of the solution. Once stirred in, this dispersion was mixed in with a hardening agent to make the final microsphere composition. These microspheres were then vacuum dried for several days, then sieved to establish a size distribution between 20 and 80 microns and sterilized by gamma irradiation prior to being implanted in rodents. SEM analysis indicated that the mean size of 50:50 microparticles was 36 microns, while 85:15 microparticles was 55 microns. Because residual methylene chloride could stimulate an unfavorable inflammatory reaction, levels were measured using gas chromatography. In both formulations, residual methylene chloride was less than 5 ppm. Microspheres were initially weighed (5 mg) and washed three times in L 15 medium (GibcoBRL, Grand Island, N.Y.). Microspheres were then pelletized and resuspended in an appropriate volume to generate a delivery of 100 microparticles/microlitre (μl) of L15 medium. The microsphere/L15 suspension was then dispersed using a 27 gauge needle prior to implantation. All animal procedures were conducted according to IACUC-approved protocols (Institutional Animal Care and Use Committee). Adult (220-300 gram) male Fisher 344 rats were anesthetized with a mixture of ketamine (65 mg/kg), and xylazine (10 mg/kg) and placed in a stereotactic frame (Stoelting, Wood Dale, Ill.). The nose bar was set 2.7 mm below the intra-aural line. A midline incision was made using a scalpel and a 500-micron hole created in the skull using a stainless steel drill bit attached to a dental drill. A 27-gauge needle was then lowered into the striatum region of the brain located at +0.2 mm Bregma, +3.0 mm lateral, 5.0 mm deep (relative to dura). Approximately 4 μl containing 100 microparticles/μl of L15 media was injected through a 27-gauge needle connected to a controlled delivery syringe pump system (PhD series pump, Harvard Apparatus, Holliston, Mass.) via polyethylene tubing and a 10 μl Hamilton Syringe (Reno, Nev.). Microparticles were injected at 1 μl/min. Following the injection, the needle was slowly withdrawn, and the overlying scalp closed with 5/0 silk suture. All animals had access to food and water ad libitum pre- and post-surgery. Animals were sacrificed via transcardial perfusion at 1 day, 2 weeks, 4 weeks, and 8 weeks post-implantation to examine microparticle bioefficacy.

Sequential 20 micron frozen tissue sections were cut and three distinct regions of rat brain analyzed along the entire length of the injection tract (cortex, upper striatum/corpus callosum, ventral striatum). Horizontal sections from each region were immunohistochemically processed for TuJ1 (immature and mature neurons), GFAP (astrocytes/glial scarring), OX-42 (resting and activated microglia), ED-1 (macrophages), and DAPI (nuclei) (see Messina et al. (2003) Experimental Neurology, 184: 816-829).

50:50 microspheres were well tolerated at both 1 and 2 months post implantation. The scarring response (GFAP) was localized to the site of injection both in the cortex and striatum, with the level of GFAP staining decreasing at 2 months. Minimal neuronal loss was evident, and the minor inflammatory reaction that was triggered at early time points (ED-1) was virtually abolished at 2 months post implantation. Excessive cellularity (DAPI) around the implant site was not noted at 2 months further suggestive of the minimal tissue reaction.

GFAP expression of 85:15 microspheres was upregulated around the injection tract suggests that at one-month post implantation, a greater inflammatory reaction had taken place. Further, neuronal loss coincided with the area of greatest GFAP reactivity. In accordance with these results, the ED-1 (macrophage) reaction was greater as evidenced by an increased number of macrophages residing along the injection tract. Notably, the inflammatory reaction was significantly reduced at two months post implantation to levels found in 50:50 microsphere-implanted animals, suggesting their long term biocompatibility is fair and appropriate.

### EXAMPLE 4

### Implantation of Cell-Coated Microspheres into the Striatum

Similar to Example 3, 50:50 microparticles (same size distribution) were generated and gamma irradiation sterilized. Microspheres were weighed (5 milligram) and washed three times in L15 medium. Microspheres were then pelletized and resuspended in growth medium that consisted of DMEM (low glucose, GibcoBRL) supplemented with 15% fetal bovine serum, pencillin-streptamycin (5 ml per 500 milliliters medium), and beta-mercaptoethanol (0.001%, Sigma, St. Louis, Mo.). The microsphere/growth medium suspension was then dispersed using a 27 gauge needle into hydrogel-coated tissue culture plates (Corning) to ensure a lack of cell attachment to the plate surface. Postpartum cells derived from umbilical tissue (see Example 2 for isolation procedure) were plated into wells containing microparticles and allowed to attach while on a rotating shaker set at low speed (5 rpm) and 37° C. After 6 hours, cultures were examined for cell attachment. Attachment was restricted to microparticles only. Plates containing cells and microparticles were then transferred to incubators at 37° C. overnight in preparation for implantation the following day (24 hours later).

Similarly to example 3, Adult (220-300 gram) male Fisher 344 rats were anesthetized with a mixture of ketamine (65 milligrams/kilogram), and xylazine (10 milligrams/kilogram) and placed in a stereotaxic frame. The nose bar was 2.7 mm below the intra-aural line. A midline incision was using a scalpel and a 500 micron hole drilled into skull using a stainless steel drill bit attached to a dental drill. A 27 gauge needle was then lowered into the striatum region of the brain at +0.2 mm Bregma, +3.0 mm lateral, 5.0 mm deep (relative to dura).

Prior to implantation, microparticles containing postpartum cells on the surface were transferred aseptically to a 15 conical tube and centrifuged 3 minutes at 800 rpm. Media supernatant was removed and microparticles containing cells were resuspended in L15 medium at a density of 100 microparticles/microliter. Approximately 4 microliters was injected through a 27 gauge needle connected to a controlled delivery syringe pump system via polyethylene tubing and a 10 microlter Hamilton syringe. Microparticles were injected at 1 microliter/minute. Following the injection, the needle was slowly withdrawn, and the overlying scalp closed with 5/0 silk suture. All animals had access to food and water ad libitum pre- and post-surgery. Animals were sacrificed at one week post-implantation to examine microparticle bioefficacy utilizing immunohistochemistry. No visible behavioral deficits were detected as a result of the implantation of microparticles containing postpartum cells on their surface.

1. A microparticle suitable for use in the treatment of a disease in a body of a mammal, comprising:
a core comprising an effective amount of a biologically active agent distributed there through; and
a cell attached to the surface of said core.

2. The microparticle of claim 1 wherein said core comprises a biocompatible, biodegradable polymer.

3. The microparticle of claim 2 wherein said polymer is selected from the group consisting of poly(lactide), poly(glycolide), poly(lactide-co-glycolide), poly(caprolactone), polycarbonates, polyamides, polyanhydrides, polyamino acids, polyortho esters, polyphosphazene, polyacetals, polycyanoacrylates, proteins, collagen, synthetic polyamino acids, prolamines, polysaccharides and heparin.

4. The microparticle of claim 1 wherein said biologically active agent is selected from the group consisting of basic fibroblast growth factor, alpha fibroblast growth factor, heparin-binding growth factor, transforming growth factor alpha or beta, epidermal growth factor, insulin derived growth factor, vascular endothelium growth factor, platelet-derived growth factor, glial growth factor, atrial natriuretic peptide, brain natriuretic peptide, glial-derived neurotrophic factor, nerve growth factor, brain-derived neurotrophic factor, platelet derived growth factor, growth differentiation factor five, morphogenetic protein 52, bone morphogenetic protein seven, bone morphogenetic protein fourteen, EPO, EPO-mimetibody, cyclosporin and a sulfamate-substituted monosaccharide.

5. The microparticle of claim 1 wherein said cell is selected from the group consisting of chondrocytes, angioblasts, myoblasts, epithelial cells, smooth muscle cells, beta cells, sertoli cells, macrophages, microglia, endothelial cells, stem cells, undifferentiated progenitor cells, pre-differentiated progenitor cells, progenitor cells, neural stem cells, neural progenitor cells, neural cells, dendritic cells and genetically transformed cells.

6. The microparticle of claim 1 further comprising a cell-adherent agent on the surface of said core.

7. The microparticle of claim 6 wherein said cell-adherent agent is derived from said cell.

8. The microparticle of claim 6 comprising a coating of said cell-adherent agent.

9. The microparticle of claim 8 wherein said cell-adherent agent is selected from the group consisting of collagen, laminin, fibronectin, gelatin, allylamines, self-assembling peptides and peptide derivatives.

10. The microparticle of claim 1 wherein said core comprises from about 0.1 to about 30 percent by weight of said biologically active agent.

11. The microparticle of claim 1 having an average diameter of from less than about 80 microns up to about 500 microns.

12. The microparticle of claim 1 having an average diameter of less than about 200 microns.

13. The microparticle of claim 1 wherein said biologically active agent is released from said core in a controlled sustained release profile.

14. The microparticle of claim 1 wherein said biologically active agent is released from said core over a period of from at least about 24 hours up to about 2 years.

15. The microparticle of claim 1 wherein said cell is viable in said body for at least about 24 hours after placement of said microparticle in said body.

16. A composition suitable for use in the treatment of a disease in a region of a body of a mammal, said composition comprising:
a plurality of microparticles suitable for use in the treatment of said disease in said region of said body of said mammal, said microparticles comprising
a core comprising an effective amount of a biologically active agent distributed there through, and
a cell attached to the surface of said core; and
a biocompatible pharmaceutically acceptable vehicle for said microparticles.

17. The composition of claim 16 wherein said vehicle for said microparticles comprises an aqueous solution.

18. A method for treatment of a disease in a region of a body of a mammal, said method comprising delivering to said region of said mammal microparticles suitable for use in the treatment of said disease, said microparticles comprising,
a core comprising an effective amount of a biologically active agent; and
a cell attached to the surface of said core.

19. The method of claim 18 wherein said microparticles are delivered invasively or non-invasively.

20. The method of claim 18 wherein said microparticles are delivered in a composition comprising said microparticles and a biocompatible pharmaceutically acceptable vehicle for said microparticles.
